(19) United States
(12) Patent Application Publication
(10) Pub. No.: US 2004/0247698 A1
(43) Pub. Date: Dec. 9, 2004

(54) Erectile Dysfunction Treatment

(76) Inventor: Carmen Maria Valenzuela Cortes, Santiago (CL)

Correspondence Address: SMITH & HOPEN PA, 15950 BAY VISTA DRIVE, SUITE 220, CLEARWATER, FL, 33760

(21) Appl. No.: 10/250,103

(22) Filed: Jun. 4, 2003

(51) Int. Cl. A61K 35/78

(52) U.S. Cl. 424/725

(57) ABSTRACT

A method for extracting compositions from a natural product, their pharmaceutical compositions, the pharmaceutical compounds, and their therapeutic uses as a sexual performance enhancer, in the treatment of erectile dysfunction, and in the treatment of hypertension in humans and animals.

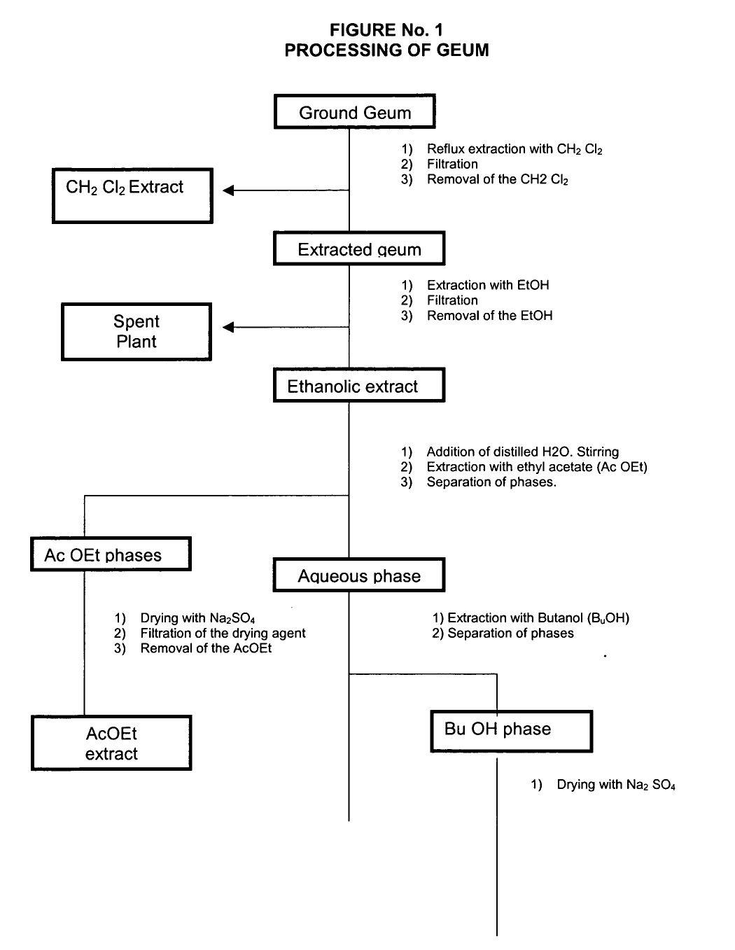

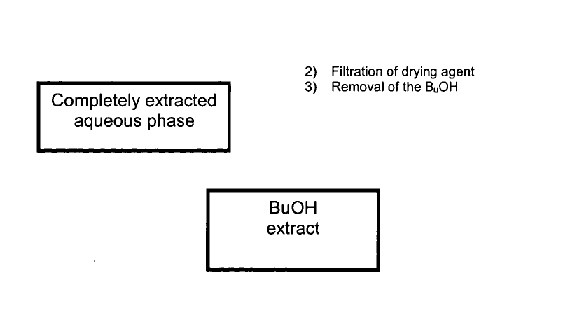

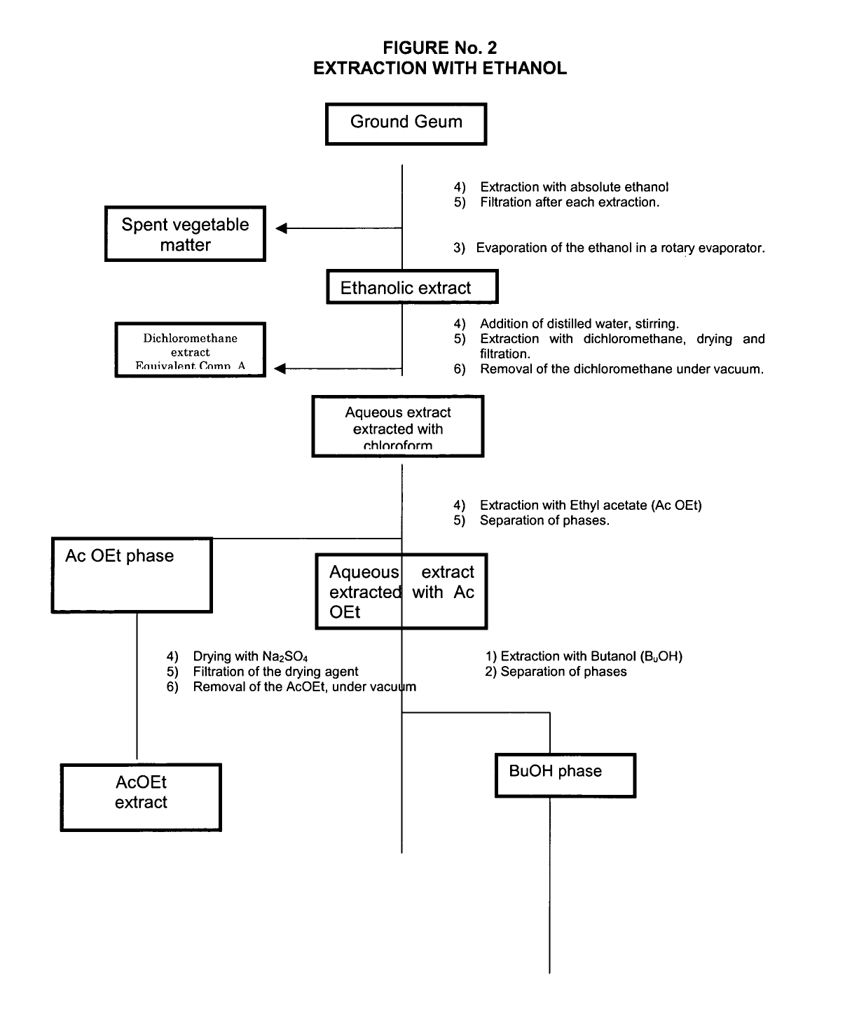

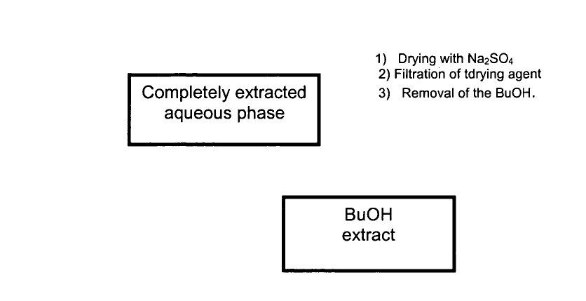

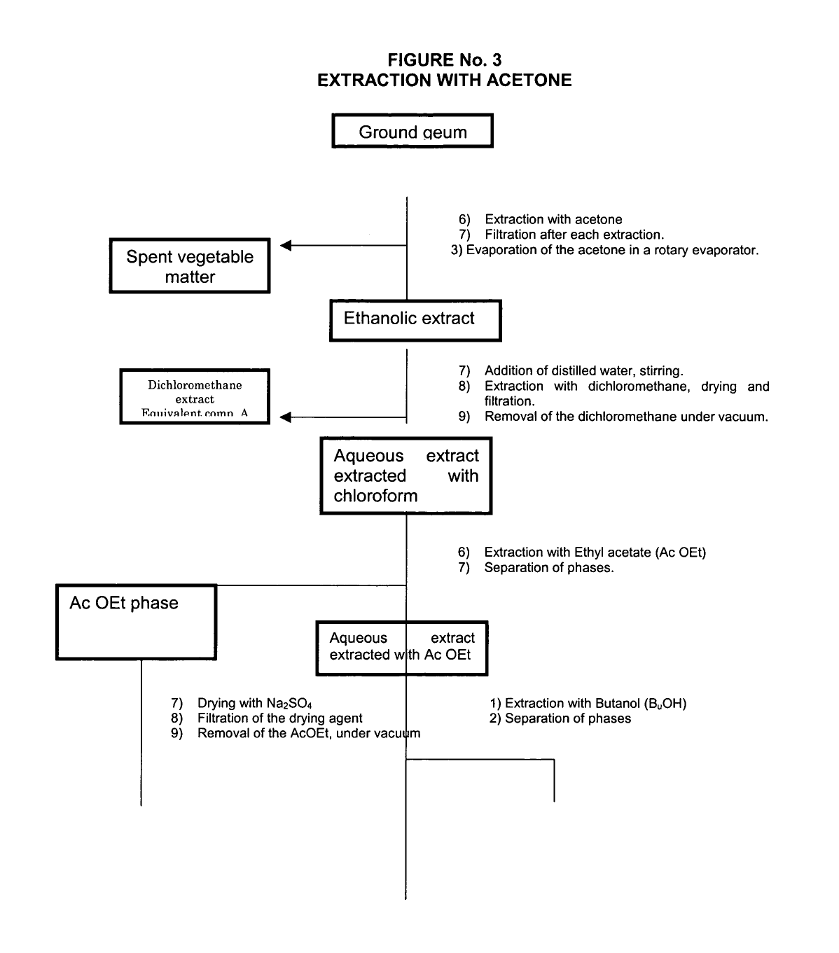

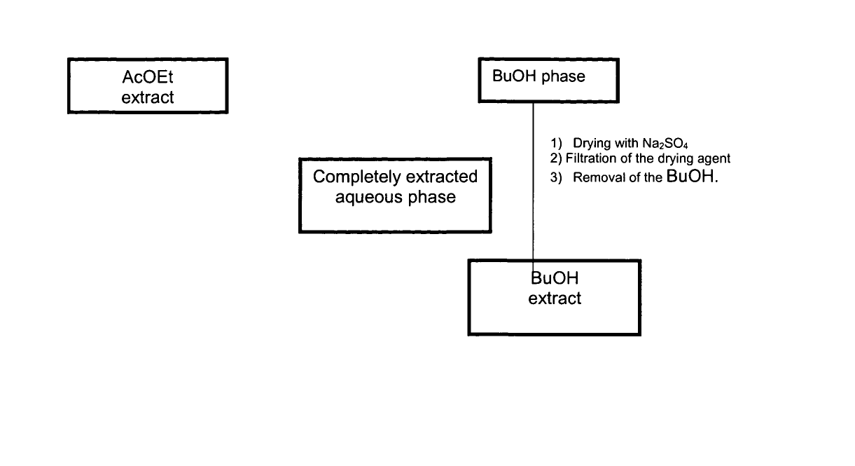

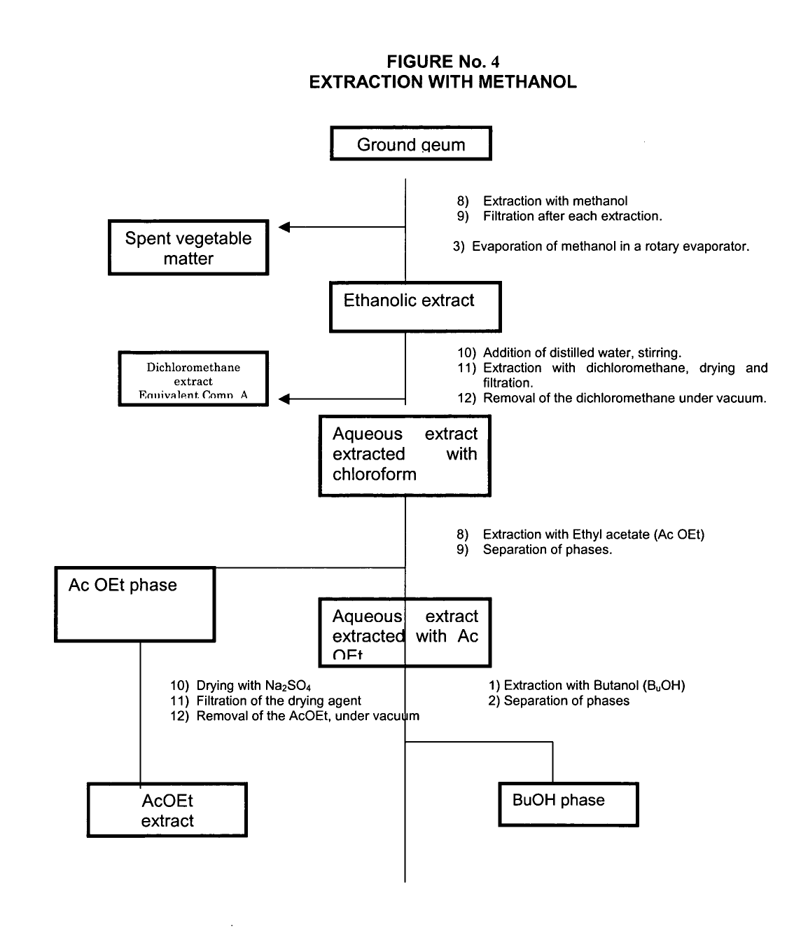

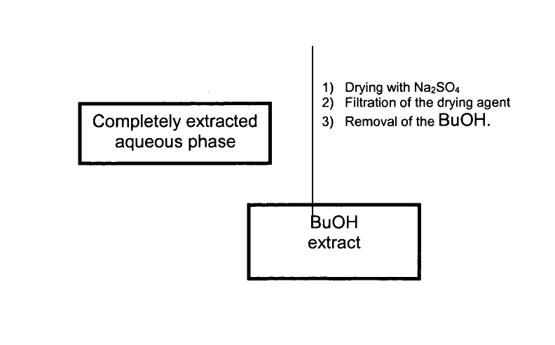

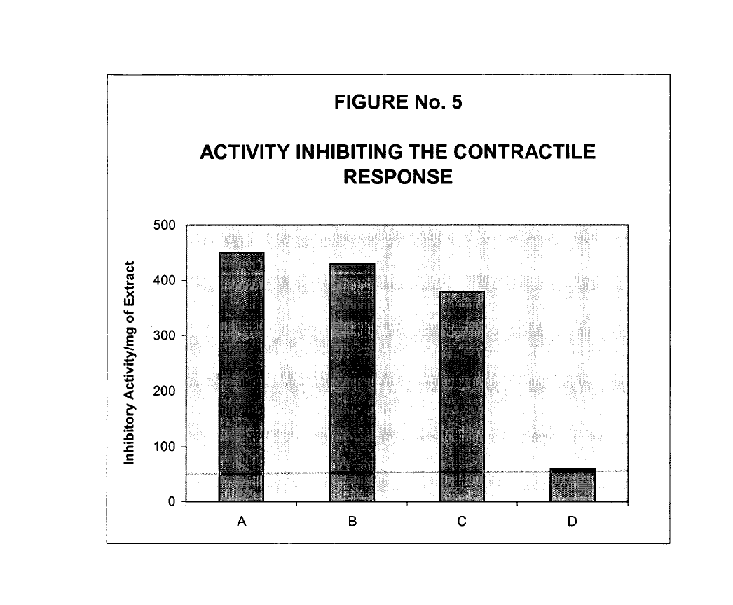

### FIELD OF THE INVENTION

This invention relates to procedures for extracting compositions from a natural product, their pharmaceutical compositions, the pharmaceutical compounds, and their therapeutic uses as a sexual performance enhancer, in the treatment of erectile dysfunction, and in the treatment of hypertension in humans and animals.

### BACKGROUND OF THE INVENTION

In popular medicine, plants of the genus Geum are associated with various therapeutic properties. For example, uses in the treatment of dental neuralgia, as appetite enhancers and emmenagogues, to facilitate menstruation, etc. are described for Geum Quellyon Sweet (Geum Chiloensis Balb.), popularly known as “Chilean avens” or “Llallante” (Juan Zin S. “La salud por medio de las plantas medicinales” [“Health through Medicinal Plants”] Ed. Don Bosco 7th Ed.1998). The same uses are described for Geum magellanicum Pers., Geum andicola (Phil) Reiche, or “Quillón.” Antimicrobial and antifungal uses have also been described.

This invention describes a procedure for obtaining extracts from plants of the genus Geum, the pharmaceutical compositions that comprise the extracts obtained, and the pharmaceutical compounds from said compositions as well as their therapeutic uses as sexual performance enhancers, in the treatment of erectile dysfunction, and in the treatment of hypertension in humans and animals.

Various synthetic pharmaceutical products for the treatment of erectile dysfunction are now on the market, but they all have side effects. For example, their use is contraindicated for patients with hypertension or cardiac defects, for older adults, etc. The compositions of this invention are from a natural source, are not toxic, and are used to prepare pharmaceutical compositions intended to treat erectile dysfunction, while also having hypotensive properties, thus avoiding all the undesirable side effects and restrictions that occur with prior art products. With respect to toxicity, it is known that the plant, when processed under prior grinding techniques, has significant concentrations of eugenol and methyl eugenol, which are phenylpropane compounds related to the known carcinogen safrole. However, in the extracts of this invention, vacuum-dried at a constant weight, these potentially carcinogenic volatile compounds are eliminated, yielding a significant technological advantage.

Another noteworthy advantage is that the activity of the extract's derivatives shows greater potency than an infusion; in other words, the fractions obtained through the use of solvents significantly exceed the pharmacological activity of the extracts with respect to an aqueous infusion.

### DESCRIPTION OF THE INVENTION

In this invention, procedures are described for obtaining extracts from plants of the genus Geum, pharmaceutical compositions that comprise the extracts obtained and the pharmaceutical compounds from said compositions, and their therapeutic uses as sexual performance enhancers, in the treatment of erectile dysfunction, and in the treatment of hypertension in humans and animals.

To obtain the pharmaceutical extracts of this invention, the whole Geum plant is used, with the root being preferable since it exhibits a more stable chemical composition.

This invention describes a procedure for obtaining compositions from plants of the genus Geum. Under this procedure, the entire plant is ground in a blade grinder and the vegetable matter is suspended in analytical grade dichloromethane and is extracted by reflux extraction; the suspension is filtered and the procedure is repeated until total extraction has been achieved. Then a residue (composition A) is obtained by evaporating the collected extracts. Following this, the vegetable matter which was extracted with dichloromethane is extracted with analytical grade ethanol, with stirring, and the procedure is repeated until an extract is obtained. A viscid syrup (composition B) is obtained by evaporation of the collected ethanolic extracts. Then composition B is mixed with chilled water to prevent decomposition and the mixture is stirred. The suspension obtained is extracted with analytical grade ethyl acetate. The ethyl acetate extracts are collected and dried; the suspension is filtered and washed; and a residue (composition C) is obtained by evaporation of the extract. Lastly, the aqueous phase is extracted with analytical grade butanol, and the butanolic extracts are processed in the same manner as those of ethyl acetate, obtaining composition D.

To obtain the fractions that are of interest, further extractions are carried out with other solvents such as water, water-ethanol, ethanol, acetone, acetone-water, methanol, methanol-water, and supercritical fluid. In addition, extracts equivalent to compositions A, B, C, and D are obtained by freeze drying or evaporation of the solvents.

### EXAMPLE 1

Geum root with equilibrium moisture content is ground (FIG. No. 1) in a blade grinder. The vegetable matter is suspended in analytical grade dichloromethane and extracted by reflux extraction for 12 to 48 hours, preferably between 16 to 40 hours, preferably yet between 20 and 30 hours, and most preferably for 24 hours. The suspension is filtered and the procedure is repeated at least four more times, preferably another three times. Through vacuum evaporation of the collected extracts (rotary evaporator), between 0.1% and 5.0% w/w, preferably between 0.5%-2.0% w/w, is obtained as a residue (extract of dichloromethane), which henceforth will be referred to as composition A (FIG. No. 1). The vegetable matter fully extracted with dichloromethane is extracted with analytical grade ethanol through magnetic stirring for 36 to 56 hours, preferably between 42 and 52 hours and most preferably 48 hours, and the procedure is repeated another two to five times, preferably between two and three times and most preferably two times, until a colorless extract is obtained. The vegetable matter completely extracted with ethanol is discarded. Through vacuum evaporation of the collected ethanolic extracts, a viscid syrup is obtained (approximate yield of 15%-45% w/w with respect to the initial material, preferably between 15-25% w/w) once completely evaporated. This, which henceforth will be referred to as composition B, is mixed with chilled water to prevent decomposition and is stirred for 12 to 36 hours, preferably for 20 to 28 hours and most preferably for 24 hours.¹ The suspension obtained is extracted with analytical grade ethyl acetate between three and five times, most preferably five times. The ethyl acetate extracts are collected and are dried with anhydrous sodium sulfate for 6 to 24 hours, preferably between 10 and 20 hours, most preferably 12 hours; the suspension is filtered and the sulfate is washed with clean ethyl acetate. ¹ [Translator's note: This is a particularly awkward sentence in the Spanish original.]

By evaporating the extract under vacuum and after keeping it in a vacuum desiccator until a constant weight is attained, a residue is obtained (yield between 2-15% w/w, preferably between 5-10% w/w), henceforth referred to as composition C. The aqueous phase (fully extracted with ethyl acetate) is extracted with analytical grade butanol until full extraction has been achieved. The butanolic extracts are processed in the same manner as the ethyl acetate extracts to yield approximately 1% to 6% by weight, preferably between 2-4% by weight, of that which we will henceforth call composition D (FIG. No. 1).

### EXMAPLE 2

Acid Hydrolysis of Composition D:

A solution of composition D (500 mg) in 5% H2SO4 (200 ml) is heated in a boiling H₂O bath for 12 hours. After cooling the reaction mixture, it is extracted with ethyl acetate. The compounds that are soluble in ethyl acetate are studied by TLC, demonstrating that they are made up of at least three compounds. Through preparative TLC, the mixture is separated to yield gallic acid, egallic acid, and valonate dilactone². Glucose is identified from the aqueous phase. ² [Translator's note: It is unclear what is meant. The Spanish reads “valonatodilactona.” In the same context elsewhere in the text, the term “valoneic acid lactone” is used.]

### EXAMPLE 3

Partial Hydrolosis of Composition D:

A solution of composition D (65 mg) in 1 mL of acetone and 25 mL of H₂O is heated under reflux for 12 hours. After cooling the reaction mixture, it is extracted with ethyl acetate. The compounds that are soluble in ethyl acetate are studied by TLC, showing the presence of at least three compounds.

To obtain the fractions of interest, further extractions are carried out with other solvents: water, water-ethanol, ethanol, acetone, acetone-water, methanol, methanol-water, and supercritical fluid. Extracts equivalent to composition B are obtained by freeze-drying or evaporating the solvents in a rotary evaporator. This composition undergoes processing identical to that described above to obtain compositions equivalent to compositions A, C, and D.

Going into greater detail, the methodologies of the above-described processes are as follows.

### EXAMPLE 4

Extraction with Water:

250 grams of Geum with equilibrium moisture content are ground and suspended in cold distilled water to prevent decomposition for 20 to 28 hours, preferably 24 hours, to guarantee stability. The suspension is filtered and the aqueous extract undergoes successive extractions with dichloromethane, ethyl acetate, and butanol. Compositions equivalent in yield and quality to compositions A, C, and D are obtained from each of these extracts.

In another experiment, the aqueous extract is freeze-dried, yielding a composition equivalent to composition B through processing of the freeze-dried extract, dissolution in water, and successive extractions with dichloromethane, ethyl acetate, and butanol.

Compositions equivalent in yield and quality to compositions A, C, and D are obtained in each of these extracts.

### EXAMPLE 5

Acetone—Water Extraction:

250 grams of Geum with equilibrium moisture content are ground and extracted by maceration with mixtures of acetone—water of various compositions for 12 to 48 hours, preferably 20 to 28 hours, most preferably 24 hours. The suspension is filtered and the acetone is removed under vacuum in a rotary evaporator, and the aqueous extract is successively extracted with dichloromethane, ethyl acetate, and butanol. Compositions equivalent in yield and quality to compositions A, C, and D are obtained in each of these extracts.

### EXAMPLE 6

Acetone Extraction:

250 grams of Geum with equilibrium moisture content are ground and extracted with analytical grade acetone for 16 to 72 hours, with magnetic stirring. The procedure is repeated until the plant is spent. By vacuum evaporation of the collected acetonic extracts, a solid with the appearance of a powder is obtained (yield in the range of 15-35%, depending on the material).

### EXAMPLE 7

Ethanol—Water Extraction:

250 grams of Geum with equilibrium moisture content are ground and extracted by maceration with mixtures of ethanol—water of various compositions for 12 to 48 hours, preferably between 20 and 28 hours, most preferably 24 hours. The suspension is filtered and the ethanol is removed under vacuum in a rotary evaporator; the aqueous extract is successively extracted with dichloromethane, ethyl acetate, and butanol. Compositions equivalent in yield and quality to compositions A, C, and D are obtained in each of these extracts.

### EXAMPLE 8

Ethanol Extraction:

250 grams of Geum with equilibrium moisture content are ground and extracted with analytical grade ethanol for 16 to 72 hours, with magnetic stirring. The procedure is repeated until the plant is spent. By vacuum evaporation of the collected ethanolic extracts, a solid with the appearance of a powder is obtained (yield in the range of 15-35%, depending on the material).

### EXAMPLE 9

Methanol Extraction:

250 grams of Geum with equilibrium moisture content are ground and extracted with analytical grade methanol for 16 to 72 hours, with magnetic stirring. The procedure is repeated until the plant is spent. By vacuum evaporation of the collected methanolic extracts, a solid with the appearance of a powder is obtained (yield in the range of 15-35%, depending on the material).

### EXAMPLE 10

Methanol—Water Extraction:

250 grams of Geum with equilibrium moisture content are ground and extracted by maceration with methanol-water mixtures of various compositions for 12 to 48 hours, preferably 20 to 28 hours, most preferably 24 hours. The suspension is filtered and the methanol is removed under vacuum in a rotary evaporator; the aqueous extract is successively extracted with dichloromethane, ethyl acetate, and butanol. Compositions equivalent in yield and quality to compositions A, C, and D are obtained in each of these extracts.

EXAMPLE 11

Extraction Using Supercritical Fluid:

250 grams of Geum with equilibrium moisture content are ground and extracted with liquid carbon dioxide in laboratory-scale equipment. The extract obtained is equivalent to composition B; after suspending it in water, it is successively extracted with dichloromethane, ethyl acetate, and butanol. Compositions equivalent in yield and quality to compositions A, C, and D are obtained in each of these extracts.

The ground Geum plant preferably contains relative moisture of 10.2% to 12.1% w/w; nitrogen, potassium, calcium, magnesium, chloride, and sodium between 2.7% and 3.1% w/w; heavy metals: zinc, copper, manganese, iron, boron, molybdenum, vanadium, cobalt, selenium, cadmium, stannum, nickel, mercury, and lead, in ppm under the toxic limit; sucrose, glucose, and fructose between 7.5% and 9.1% w/w; composition A between 0.5% and 2.0% w/w; composition B between 15% and 25% w/w; composition C between 5% and 10% w/w; composition D between 2% and 4% w/w.

The pharmaceutical compositions contained in the composition A, B, C, and D extracts are described below. Said compositions do not have the toxicity problems related to the eugenol and methyl eugenol compounds—these being molecules with carcinogenic potential (de Vicenzi et al., 2000) found in the ground plant—since those compounds are not present in the extracts contained in compositions A, B, C and D. This is the case because the extracts of this invention are vacuum-dried at a constant weight, thus eliminating the aforementioned volatile compounds. This is demonstrated in the chromatographic and spectroscopic studies (TLC, GLC-EM), which also clarify the A, B, C, and D compositions with greater precision.

The chemical components of compositions A, B, C, and D are obtained through various techniques, principally chromatographic and spectroscopic techniques, of which the results are summarized below:

The extract contained in composition A is analyzed by thin layer chromatography and gas chromatography coupled with mass spectrometry, with the following compounds being found:

a) Hydrocarbons C₂₃H₄₈ and C₂₅H₅₂, for example 6-tridecene and 2,6-dimethylheptadecane, and/or

b) Aromatic compounds such as benzophenone, 1,2-dihydro-1-phenylnaphthalene, (1,3-dimethyl-3-butenyl)-benzene, scopoletine, ligustilide, cinnamaldehyde, and benzaldehyde, and/or

c) Simple sugars such as arabinitol, thritol³, and glucitol, and/or ³ [Translator's note: The Spanish, “trietol” may be a misspelling for “tritol,” English “thritol.”The same comment applies to all cases of “thritol” in the document.]

d) Vegetable sterols or phytosterols such as β-sitosterol, campesterol, stigmasterol, brassicasterol, and related phytosterols, and/or

e) Saturated and unsaturated fatty acids such as octadecanoic acid, oleic acid, α-linolenic acid, Z,Z-9,12-octadecanoic acid, and hexadecanoic acid, and/or

f) Acyl glycerols such as glyceryl hexadecanoate, and/or

g) Pentacyclic triterpenes such as alpha amirine, beta amirine, ursolic acid, and oleanolic acid, and/or

h) Monoterpenes such as camphor, alpha-pinene, and beta-pinene.

The extract contained in composition B is analyzed by Fourier transform infrared, NMR of ¹H (300 MHz), and unidimensional and bidimensional thin layer chromatography, and the following compounds are found:

a) Carbohydrates such as sucrose, glucose, fructose, arabinose, and xylose, and/or

b) Acids such as gallic acid, and/or

c) Ellagic acid esters, and/or

d) Phenols such as 1,2,3-trihydroxybenzene, and/or

e) Flavonoids such as quercitrin and quercetin, and/or

f) Pentacyclic triterpenes such as ursolic acid and oleanolic acid, and/or g) Galloyl glucoses such as 2,4,6-tri-O-galloyl-D-glucose, 1,3,4,6-tetra-O-galloyl-β-D- glucose, 1,2,4,6-tetra-O-galloyl-β-D-glucose, 1,2,3,4,6-penta-O-galloyl-β-D-glucose and 1,2,3,4,5-penta-O-galloyl-β-D-glucose, and/or

h) Dehydro-ellagitannins that yielded gallic acid, egallic acid, valoneic acid lactone, and glucose through hydrolysis.

The extract contained in composition C is analyzed by Fourier transform infrared, NMR of ¹H (300 MHz), and unidimensional and bidimensional thin layer chromatography, and the following compounds are found:

a) Acids such as gallic acid, and/or

b) Esters such as ellagic acid ester, and/or

c) Phenols such as 1,2,3-trihydroxybenzene, and/or

d) Flavonoids such as quercitrin and quercetin, and/or

e) Pentacyclic triterpenes such as ursolic acid and oleanolic acid, and/or

f) Galloyl glucoses such as 2,4,6-tri-O-galloyl-D-glucose, 1,3,4,6-tetra-O-galloyl-β-D- glucose, 1,2,4,6-tetra-O-galloyl-β-D-glucose, 1,2,3,4,6-penta-O-galloyl-β-D-glucose and 1,2,3,4,5-penta-O-galloyl-β-D-glucose.

The extract contained in composition D is analyzed by Fourier transform infrared, NMR of ¹H (300 MHz), and thin layer chromatography and is made up of dehydro-ellagitannins that yield gallic acid, egallic acid, valoneic acid lactone, and glucose through acid hydrolysis.

The extract contained in composition B through processing in turn contains fractions A, C, and D in a mixture.

Pharmaceutical composition A principally comprises at least pentacyclic triterpenes selected from alpha amirine, beta amirine, ursolic acid, and oleanolic acid, and/or monoterpenes selected from camphor, alpha-pinene and beta-pinene. In addition, it comprises aromatic compounds selected from benzophenone, 1,2-dihydro-1-phenylnaphthalene, (1,3-dimethyl-3-butenyl)-benzene, scopoletine, ligustilide, cinnamaldehyde, and benzaldehyde, and/or hydrocarbons C₂₃H₄₈ and C₂₅H₅₂ selected from 6-tridecene and 2,6-dimethylheptadecane and/or simple sugars selected from arabinitol, thritol and glucitol, and/or vegetable sterols or phytosterols selected from β-sitosterol, campesterol, stigmasterol, brassicasterol, and related phytosterols, and/or saturated and unsaturated fatty acids selected from octadecanoic acid, oleic acid, α-linolenic acid, Z,Z-9,12-octadecanoic acid, and hexadecanoic acid, and/or acyl glycerols such as glyceryl hexadecanoate.

### EXAMPLE 12

Composition A obtained through the above-described procedure contains approximately 30%-50% (preferably about 40%) hydrocarbons, and/or approximately 0.2%-1% (preferably about 0.5%) aromatic compounds, and/or approximately 10%-20% (preferably about 30%) in sugars, saturated and unsaturated fatty acids, acyl glycerols, and/or approximately 1%-3% (preferably about 2%) pentacyclic triterpenes.

Pharmaceutical compound B comprises principally at least gallic acid, associated salts, and/or esters. In addition, it comprises ellagic acid esters, and/or dehydro-ellagitannins that are precursors of gallic acid, egallic acid, valoneic acid lactone and glucose, and/or pentacyclic triterpenes selected from ursolic acid and/or oleanolic acid, and/or phenols such as 1,2,3-trihydroxybenzene, and/or carbohydrates selected from sucrose, glucose, fructose, arabinose and xylose, and/or flavonoids selected from quercitrin and quercetin, and/or galloyl glucoses selected from 2,4,6-tri-O-galloyl-D-glucose, 1,3,4,6-tetra-O-galloyl-β-D-glucose, 1,2,4,6-tetra-O-galloyl-β-D-glucose, 1,2,3,4,6-penta-O-galloyl-β-D-glucose and 1,2,3,4,5-penta-O-galloyl-β-D-glucose.

### EXAMPLE 13

Composition B obtained through the above-described procedure contains approximately 30%-50% (preferably about 40%) carbohydrates, and/or approximately 1%-3% (preferably about 2%) gallic acid, and/or approximately 1%-3% (preferably about 2%) ellagic acid esters, and/or approximately 0.2%-1% (preferably about 0.5%) phenols, and/or approximately 2.5%-4.5% (preferably about 3.5%) flavonoids, and/or approximately 1%-3% (preferably about 2%) pentacyclic triterpenes, and/or approximately 20%-40% (preferably about 30%) galloyl glucoses, and/or approximately 10%-30% (preferably about 20%) dehydro-ellagitannins.

Pharmaceutical composition C principally comprises at least gallic acid. In addition, it comprises ellagic acid esters, and/or pentacyclic triterpenes selected from ursolic acid and/or oleanolic acid, and/or phenols such as 1,2,3-trihydroxybenzene, and/or flavonoids selected from quercitrin and quercetin, galloyl glucoses selected from 2,4,6-tri-O-galloyl-D-glucose, 1,3,4,6-tetra-O-galloyl-β-D-glucose, 1,2,4,6-tetra-O-galloyl-β-D-glucose, 1,2,3,4,6-penta-O-galloyl-β-D-glucose, and 1,2,3,4,5-penta-O-galloyl-β-D-glucose.

### EXMAPLE 14

Composition C obtained through the above-described procedure contains approximately 1%-3% (preferably about 2%) gallic acid, and/or approximately 1%-3% (preferably about 2%) ellagic acid esters, and/or approximately 0.2%-1% (preferably about 0.5%) phenols, and/or approximately 2.5%-4.5% (preferably about 3.5%) flavonoids, and/or approximately 1%-3% (preferably 2%) pentacyclic triterpenes, and/or approximately 20%-40% (preferably about 30%) galloyl glucoses.

Pharmaceutical composition D comprises at least approximately 10% to 30% dehydro-ellagitannins that are precursors of gallic acid, egallic acid, valoneic acid lactone, and glucose, preferably about 20%.

In addition, pharmaceutical compositions A, B, C and D comprise pyrogallol, ethyl gallate, methyl gallate, triacetylgallic acid, propyl gallate, octyl gallate, 3,4,5-trimethoxybenzoic acid, and tannic acid.

This invention also describes the compounds contained in the pharmaceutical compositions obtained from the extracts, which comprise compounds corresponding to the general formula:

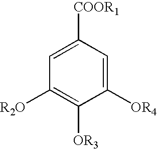

where R1 represents a group selected from acetyl; hydrogen; propionyl; butanoyl; pentanoyl; hexanoyl; heptanoyl; benzoyl; or substituted benzoyl with a nitro, chlorine, bromine, fluorine, methoxyl, methyl, hydroxyl, or acetyl group; methyl; ethyl; propyl; butyl; pentyl; hexyl; heptyl; benzyl, or substituted benzyl with a nitro, chlorine, bromine, fluorine, methoxyl, methyl, hydroxyl or acetyl group; and R2, R3, and/or R4 represent a group selected from acetyl; hydrogen, propionyl; butanoyl; pentanoyl; hexanoyl; heptanoyl; benzoyl, or substituted benzoyl with a nitro, chlorine, bromine, fluorine, methoxyl, methyl, hydroxyl or acetyl group; methyl; ethyl; propyl; butyl; pentyl; hexyl; heptyl; benzyl; or substituted benzyl with a nitro, chlorine, bromine, fluorine, methoxyl, methyl, hydroxyl, or acetyl group, and their preparation procedures. More specifically, merely by way of illustration and without limitation, the compounds are as follows:

Triacetylgallic acid, tripropionylgallic acid, tributanoylgallic acid, tripentanoylgallic acid, tribenzoylgallic acid, trihexanoylgallic acid, triheptanoylgallic acid, 2-nitro tribenzoylgallic acid, 3-nitro tribenzoylgallic acid, 4-nitro tribenzoylgallic acid, 2-chloro tribenzoylgallic acid, 3-chloro tribenzoylgallic acid, 4-chloro tribenzoylgallic acid, 2-bromo tribenzoylgallic acid, 3-bromo tribenzoylgallic acid, 4-bromo tribenzoylgallic acid, 2-fluoro tribenzoylgallic acid, 3-fluoro tribenzoylgallic acid, 4-fluoro tribenzoylgallic acid, 2-methoxy tribenzoylgallic acid, 3-methoxy tribenzoylgallic acid, 4-methoxy tribenzoylgallic acid, 2-hydroxy tribenzoylgallic acid, 3-hydroxy tribenzoylgallic acid, 4-hydroxy tribenzoylgallic acid, 2-methyl tribenzoylgallic acid, 3-methyl tribenzoylgallic acid, 4-methyl tribenzoylgallic acid, 2-acetyl tribenzoylgallic acid, 3-acetyl tribenzoylgallic acid, 4-acetyl tribenzoylgallic acid.

Trimethylgallic acid, tripropylgallic acid, tributylgallic acid, tripentylgallic acid, tribenzylgallic acid, trihexylgallic acid, triheptylgallic acid, 2-nitro tribenzylgallic acid, 3-nitro tribenzylgallic acid, 4-nitro tribenzylgallic acid, 2-chloro tribenzylgallic acid, 3-chloro tribenzylgallic acid, 4-chloro tribenzylgallic acid, 2-bromo tribenzylgallic acid, 3-bromo tribenzylgallic acid, 4-bromo tribenzylgallic acid, 2-fluoro tribenzylgallic acid, 3-fluoro tribenzylgallic acid, 4-fluoro tribenzylgallic acid, 2-methoxy tribenzylgallic acid, 3-methoxy tribenzylgallic acid, 4-methoxy tribenzylgallic acid, 2-hydroxy tribenzylgallic acid, 3-hydroxy tribenzylgallic acid, 4-hydroxy tribenzylgallic acid, 2-methyl tribenzylgallic acid, 3-methyl tribenzylgallic acid, 4-methyl tribenzylgallic acid, 2-acetyl tribenzylgallic acid, 3-acetyl tribenzylgallic acid, 4-acetyl tribenzylgallic acid.

Methyl triacetylgallate, ethyl triacetylgallate, propyl triacetylgallate, butyl triacetylgallate, pentyl triacetylgallate, benzyl triacetylgallate, 2-nitro benzyl triacetylgallate, 3-nitro benzyl triacetylgallate, 4-nitro benzyl triacetylgallate, 2-chloro benzyl triacetylgallate, 3-chloro benzyl triacetylgallate, 4-chloro benzyl triacetylgallate, 2-bromo benzyl triacetylgallate, 3-bromo benzyl triacetylgallate, 4-bromo benzyl triacetylgallate, 2-fluoro benzyl triacetylgallate, 3-fluoro benzyl triacetylgallate, 4-fluoro benzyl triacetylgallate, 2-methoxy benzyl triacetylgallate, 3-methoxy benzyl triacetylgallate, 4-methoxy benzyl triacetylgallate, 2-hydroxy benzyl triacetylgallate, 3-hydroxy benzyl triacetylgallate, 4-hydroxy benzyl triacetylgallate, 2-methyl benzyl triacetylgallate, 3-methyl benzyl triacetylgallate, 4-methyl benzyl triacetylgallate, 2-acetyl benzyl triacetylgallate, 3-acetyl benzyl triacetylgallate, 4-acetyl benzyl triacetylgallate.

Methyl tripropionylgallate, ethyl tripropionylgallate, propyl tripropionylgallate, butyl tripropionylgallate, pentyl tripropionylgallate, benzyl tripropionylgallate, 2-nitro benzyl tripropionylgallate, 3-nitro benzyl tripropionylgallate, 4-nitro benzyl tripropionylgallate, 2-chloro benzyl tripropionylgallate, 3-chloro benzyl tripropionylgallate, 4-chloro benzyl tripropionylgallate, 2-bromo benzyl tripropionylgallate, 3-bromo benzyl tripropionylgallate, 4-bromo benzyl tripropionylgallate, 2-fluoro benzyl tripropionylgallate, 3-fluoro benzyl tripropionylgallate, 4-fluoro benzyl tripropionylgallate, 2-methoxy benzyl tripropionylgallate, 3-methoxy benzyl tripropionylgallate, 4-methoxy benzyl tripropionylgallate, 2-hydroxy benzyl tripropionylgallate, 3-hydroxy benzyl tripropionylgallate, 4-hydroxy benzyl tripropionylgallate, 2-methyl benzyl tripropionylgallate, 3-methyl benzyl tripropionylgallate, 4-methyl benzyl tripropionylgallate, 2-acetyl benzyl tripropionylgallate, 3-acetyl benzyl tripropionylgallate, 4-acetyl benzyl tripropionylgallate.

Methyl tributanoylgallate, ethyl tributanoylgallate, propyl tributanoylgallate, butyl tributanoylgallate, pentyl tributanoylgallate, benzyl tributanoylgallate, 2-nitro benzyl tributanoylgallate, 3-nitro benzyl tributanoylgallate, 4-nitro benzyl tributanoylgallate, 2-chloro benzyl tributanoylgallate, 3-chloro benzyl tributanoylgallate, 4-chloro benzyl tributanoylgallate, 2-bromo benzyl tributanoylgallate, 3-bromo benzyl tributanoylgallate, 4-bromo benzyl tributanoylgallate, 2-fluoro benzyl tributanoylgallate, 3-fluoro benzyl tributanoylgallate, 4-fluoro benzyl tributanoylgallate, 2-methoxy benzyl tributanoylgallate, 3-methoxy benzyl tributanoylgallate, 4-methoxy benzyl tributanoylgallate, 2-hydroxy benzyl tributanoylgallate, 3-hydroxy benzyl tributanoylgallate, 4-hydroxy benzyl tributanoylgallate, 2-methyl benzyl tributanoylgallate, 3-methyl benzyl tributanoylgallate, 4-methyl benzyl tributanoylgallate, 2-acetyl benzyl tributanoylgallate, 3-acetyl benzyl tributanoylgallate, 4-acetyl benzyl tributanoylgallate.

Methyl tripentanoylgallate, ethyl tripentanoylgallate, propyl tripentanoylgallate, butyl tripentanoylgallate, pentyl tripentanoylgallate, benzyl tripentanoylgallate, 2-nitro benzyl tripentanoylgallate, 3-nitro benzyl tripentanoylgallate, 4-nitro benzyl tripentanoylgallate, 2-chloro benzyl tripentanoylgallate, 3-chloro benzyl tripentanoylgallate, 4-chloro benzyl tripentanoylgallate, 2-bromo benzyl tripentanoylgallate, 3-bromo benzyl tripentanoylgallate, 4-bromo benzyl tripentanoylgallate, 2-fluoro benzyl tripentanoylgallate, 3-fluoro benzyl tripentanoylgallate, 4-fluoro benzyl tripentanoylgallate, 2-methoxy benzyl tripentanoylgallate, 3-methoxy benzyl tripentanoylgallate, 4-methoxy benzyl tripentanoylgallate, 2-hydroxy benzyl tripentanoylgallate, 3-hydroxy benzyl tripentanoylgallate, 4-hydroxy benzyl tripentanoylgallate, 2-methyl benzyl tripentanoylgallate, 3-methyl benzyl tripentanoylgallate, 4-methyl benzyl tripentanoylgallate, 2-acetyl benzyl tripentanoylgallate, 3-acetyl benzyl tripentanoylgallate, 4-acetyl benzyl tripentanoylgallate.

Methyl tribenzoylgallate, ethyl tribenzoylgallate, propyl tribenzoylgallate, butyl tribenzoylgallate, pentyl tribenzoylgallate, benzyl tribenzoylgallate, 2-nitro benzyl tribenzoylgallate, 3-nitro benzyl tribenzoylgallate, 4-nitro benzyl tribenzoylgallate, 2-chloro benzyl tribenzoylgallate, 3-chloro benzyl tribenzoylgallate, 4-chloro benzyl tribenzoylgallate, 2-bromo benzyl tribenzoylgallate, 3-bromo benzyl tribenzoylgallate, 4-bromo benzyl tribenzoylgallate, 2-fluoro benzyl tribenzoylgallate, 3-fluoro benzyl tribenzoylgallate, 4-fluoro benzyl tribenzoylgallate, 2-methoxy benzyl tribenzoylgallate, 3-methoxy benzyl tribenzoylgallate, 4-methoxy benzyl tribenzoylgallate, 2-hydroxy benzyl tribenzoylgallate, 3-hydroxy benzyl tribenzoylgallate, 4-hydroxy benzyl tribenzoylgallate, 2-methyl benzyl tribenzoylgallate, 3-methyl benzyl tribenzoylgallate, 4-methyl benzyl tribenzoylgallate, 2-acetyl benzyl tribenzoylgallate, 3-acetyl benzyl tribenzoylgallate, 4-acetyl benzyl tribenzoylgallate.

Methyl tribenzoylgallate, ethyl tribenzoylgallate, propyl tribenzoylgallate, butyl tribenzoylgallate, pentyl tribenzoylgallate, benzyl tribenzoylgallate, 2-nitro benzyl tribenzoylgallate, 3-nitro benzyl tribenzoylgallate, 4-nitro benzyl tribenzoylgallate, 2-chloro benzyl tribenzoylgallate, 3-chloro benzyl tribenzoylgallate, 4-chloro benzyl tribenzoylgallate, 2-bromo benzyl tribenzoylgallate, 3-bromo benzyl tribenzoylgallate, 4-bromo benzyl tribenzoylgallate, 2-fluoro benzyl tribenzoylgallate, 3-fluoro benzyl tribenzoylgallate, 4-fluoro benzyl tribenzoylgallate, 2-methoxy benzyl tribenzoylgallate, 3-methoxy benzyl tribenzoylgallate, 4-methoxy benzyl tribenzoylgallate, 2-hydroxy benzyl tribenzoylgallate, 3-hydroxy benzyl tribenzoylgallate, 4-hydroxy benzyl tribenzoylgallate, 2-methyl benzyl tribenzoylgallate, 3-methyl benzyl tribenzoylgallate, 4-methyl benzyl tribenzoylgallate, 2-acetyl benzyl tribenzoylgallate, 3-acetyl benzyl tribenzoylgallate, 4-acetyl benzyl tribenzoylgallate.

Methyl trihexanoylgallate, ethyl trihexanoylgallate, propyl trihexanoylgallate, butyl trihexanoylgallate, pentyl trihexanoylgallate, benzyl trihexanoylgallate, 2-nitro benzyl trihexanoylgallate, 3-nitro benzyl trihexanoylgallate, 4-nitro benzyl trihexanoylgallate, 2-chloro benzyl trihexanoylgallate, 3-chloro benzyl trihexanoylgallate, 4-chloro benzyl trihexanoylgallate, 2-bromo benzyl trihexanoylgallate, 3-bromo benzyl trihexanoylgallate, 4-bromo benzyl trihexanoylgallate, 2-fluoro benzyl trihexanoylgallate, 3-fluoro benzyl trihexanoylgallate, 4-fluoro benzyl trihexanoylgallate, 2-methoxy benzyl trihexanoylgallate, 3-methoxy benzyl trihexanoylgallate, 4-methoxy benzyl trihexanoylgallate, 2-hydroxy benzyl trihexanoylgallate, 3-hydroxy benzyl trihexanoylgallate, 4-hydroxy benzyl trihexanoylgallate, 2-methyl benzyl trihexanoylgallate, 3-methyl benzyl trihexanoylgallate, 4-methyl benzyl trihexanoylgallate, 2-acetyl benzyl trihexanoylgallate, 3-acetyl benzyl trihexanoylgallate, 4-acetyl benzyl trihexanoylgallate.

Methyl triheptanoylgallate, ethyl triheptanoylgallate, propyl triheptanoylgallate, butyl triheptanoylgallate, pentyl triheptanoylgallate, benzyl triheptanoylgallate, 2-nitro benzyl triheptanoylgallate, 3-nitro benzyl triheptanoylgallate, 4-nitro benzyl triheptanoylgallate, 2-chloro benzyl triheptanoylgallate, 3-chloro benzyl triheptanoylgallate, 4-chloro benzyl triheptanoylgallate, 2-bromo benzyl triheptanoylgallate, 3-bromo benzyl triheptanoylgallate, 4-bromo benzyl triheptanoylgallate, 2-fluoro benzyl triheptanoylgallate, 3-fluoro benzyl triheptanoylgallate, 4-fluoro benzyl triheptanoylgallate, 2-methoxy benzyl triheptanoylgallate, 3-methoxy benzyl triheptanoylgallate, 4-methoxy benzyl triheptanoylgallate, 2-hydroxy benzyl triheptanoylgallate, 3-hydroxy benzyl triheptanoylgallate, 4-hydroxy benzyl triheptanoylgallate, 2-methyl benzyl triheptanoylgallate, 3-methyl benzyl triheptanoylgallate, 4-methyl benzyl triheptanoylgallate, 2-acetyl benzyl triheptanoylgallate, 3-acetyl benzyl triheptanoylgallate, 4-acetyl benzyl triheptanoylgallate.

Methyl 2-nitro tribenzoylgallate, methyl 3-nitro tribenzoylgallate, methyl 4-nitro tribenzoylgallate, methyl 2-chloro tribenzoylgallate, methyl 3-chloro tribenzoylgallate, methyl 4-chloro tribenzoylgallate, methyl 2-bromo tribenzoylgallate, methyl 3-bromo tribenzoylgallate, methyl 4-bromo tribenzoylgallate, methyl 2-fluoro tribenzoylgallate, methyl 3-fluoro tribenzoylgallate, methyl 4-fluoro tribenzoylgallate, methyl 2-methoxy tribenzoylgallate, methyl 3-methoxy tribenzoylgallate, methyl 4-methoxy tribenzoylgallate, methyl 2-hydroxy tribenzoylgallate, methyl 3-hydroxy tribenzoylgallate, methyl 4-hydroxy tribenzoylgallate, methyl 2-methyl tribenzoylgallate, methyl 3-methyl tribenzoylgallate, methyl 4-methyl tribenzoylgallate, methyl 2-acetyl tribenzoylgallate, methyl 3-acetyl tribenzoylgallate, methyl 4-acetyl tribenzoylgallate.

Ethyl 2-nitro tribenzoylgallate, ethyl 3-nitro tribenzoylgallate, ethyl 4-nitro tribenzoylgallate, ethyl 2-chloro tribenzoylgallate, ethyl 3-chloro tribenzoylgallate, ethyl 4-chloro tribenzoylgallate, ethyl 2-bromo tribenzoylgallate, ethyl 3-bromo tribenzoylgallate, ethyl 4-bromo tribenzoylgallate, ethyl 2-fluoro tribenzoylgallate, ethyl 3-fluoro tribenzoylgallate, ethyl 4-fluoro tribenzoylgallate, ethyl 2-methoxy tribenzoylgallate, ethyl 3-methoxy tribenzoylgallate, ethyl 4-methoxy tribenzoylgallate, ethyl 2-hydroxy tribenzoylgallate, ethyl 3-hydroxy tribenzoylgallate, ethyl 4-hydroxy tribenzoylgallate, ethyl 2-methyl tribenzoylgallate, ethyl 3-methyl tribenzoylgallate, ethyl 4-methyl tribenzoylgallate, ethyl 2-acetyl tribenzoylgallate, ethyl 3-acetyl tribenzoylgallate, ethyl 4-acetyl tribenzoylgallate.

Propyl 2-nitro tribenzoylgallate, propyl 3-nitro tribenzoylgallate, propyl 4-nitro tribenzoylgallate, propyl 2-chloro tribenzoylgallate, propyl 3-chloro tribenzoylgallate, propyl 4-chloro tribenzoylgallate, propyl 2-bromo tribenzoylgallate, propyl 3-bromo tribenzoylgallate, propyl 4-bromo tribenzoylgallate, propyl 2-fluoro tribenzoylgallate, propyl 3-fluoro tribenzoylgallate, propyl 4-fluoro tribenzoylgallate, propyl 2-methoxy tribenzoylgallate, propyl 3-methoxy tribenzoylgallate, propyl 4-methoxy tribenzoylgallate, propyl 2-hydroxy tribenzoylgallate, propyl 3-hydroxy tribenzoylgallate, propyl 4-hydroxy tribenzoylgallate, propyl 2-methyl tribenzoylgallate, propyl 3-methyl tribenzoylgallate, propyl 4-methyl tribenzoylgallate, propyl 2-acetyl tribenzoylgallate, propyl 3-acetyl tribenzoylgallate, propyl 4-acetyl tribenzoylgallate.

Butyl 2-nitro tribenzoylgallate, butyl 3-nitro tribenzoylgallate, butyl 4-nitro tribenzoylgallate, butyl 2-chloro tribenzoylgallate, butyl 3-chloro tribenzoylgallate, butyl 4-chloro tribenzoylgallate, butyl 2-bromo tribenzoylgallate, butyl 3-bromo tribenzoylgallate, butyl 4-bromo tribenzoylgallate, butyl 2-fluoro tribenzoylgallate, butyl 3-fluoro tribenzoylgallate, butyl 4-fluoro tribenzoylgallate, butyl 2-methoxy tribenzoylgallate, butyl 3-methoxy tribenzoylgallate, butyl 4-methoxy tribenzoylgallate, butyl 2-hydroxy tribenzoylgallate, butyl 3-hydroxy tribenzoylgallate, butyl 4-hydroxy tribenzoylgallate, butyl 2-methyl tribenzoylgallate, butyl 3-methyl tribenzoylgallate, butyl 4-methyl tribenzoylgallate, butyl 2-acetyl tribenzoylgallate, butyl 3-acetyl tribenzoylgallate, butyl 4-acetyl tribenzoylgallate.

Pentyl 2-nitro tribenzoylgallate, pentyl 3-nitro tribenzoylgallate, pentyl 4-nitro tribenzoylgallate, pentyl 2-chloro tribenzoylgallate, pentyl 3-chloro tribenzoylgallate, pentyl 4-chloro tribenzoylgallate, pentyl 2-bromo tribenzoylgallate, pentyl 3-bromo tribenzoylgallate, pentyl 4-bromo tribenzoylgallate, pentyl 2-fluoro tribenzoylgallate, pentyl 3-fluoro tribenzoylgallate, pentyl 4-fluoro tribenzoylgallate, pentyl 2-methoxy tribenzoylgallate, pentyl 3-methoxy tribenzoylgallate, pentyl 4-methoxy tribenzoylgallate, pentyl 2-hydroxy tribenzoylgallate, pentyl 3-hydroxy tribenzoylgallate, pentyl 4-hydroxy tribenzoylgallate, pentyl 2-methyl tribenzoylgallate, pentyl 3-methyl tribenzoylgallate, pentyl 4-methyl tribenzoylgallate, pentyl 2-acetyl tribenzoylgallate, pentyl 3-acetyl tribenzoylgallate, pentyl 4-acetyl tribenzoylgallate.

Benzyl 2-nitro tribenzoylgallate, benzyl 3-nitro tribenzoylgallate, benzyl 4-nitro tribenzoylgallate, benzyl 2-chloro tribenzoylgallate, benzyl 3-chloro tribenzoylgallate, benzyl 4-chloro tribenzoylgallate, benzyl 2-bromo tribenzoylgallate, benzyl 3-bromo tribenzoylgallate, benzyl 4-bromo tribenzoylgallate, benzyl 2-fluoro tribenzoylgallate, benzyl 3-fluoro tribenzoylgallate, benzyl 4-fluoro tribenzoylgallate, benzyl 2-methoxy tribenzoylgallate, benzyl 3-methoxy tribenzoylgallate, benzyl 4-methoxy tribenzoylgallate, benzyl 2-hydroxy tribenzoylgallate, benzyl 3-hydroxy tribenzoylgallate, benzyl 4-hydroxy tribenzoylgallate, benzyl 2-methyl tribenzoylgallate, benzyl 3-methyl tribenzoylgallate, benzyl 4-methyl tribenzoylgallate, benzyl 2-acetyl tribenzoylgallate, benzyl 3-acetyl tribenzoylgallate, benzyl 4-acetyl tribenzoylgallate.

Hexyl 2-nitro tribenzoylgallate, hexyl 3-nitro tribenzoylgallate, hexyl 4-nitro tribenzoylgallate, hexyl 2-chloro tribenzoylgallate, hexyl 3-chloro tribenzoylgallate, hexyl 4-chloro tribenzoylgallate, hexyl 2-bromo tribenzoylgallate, hexyl 3-bromo tribenzoylgallate, hexyl 4-bromo tribenzoylgallate, hexyl 2-fluoro tribenzoylgallate, hexyl 3-fluoro tribenzoylgallate, hexyl 4-fluoro tribenzoylgallate, hexyl 2-methoxy tribenzoylgallate, hexyl 3-methoxy tribenzoylgallate, hexyl 4-methoxy tribenzoylgallate, hexyl 2-hydroxy tribenzoylgallate, hexyl 3-hydroxy tribenzoylgallate, hexyl 4-hydroxy tribenzoylgallate, hexyl 2-methyl tribenzoylgallate, hexyl 3-methyl tribenzoylgallate, hexyl 4-methyl tribenzoylgallate, hexyl 2-acetyl tribenzoylgallate, hexyl 3-acetyl tribenzoylgallate, hexyl 4-acetyl tribenzoylgallate.

Heptyl 2-nitro tribenzoylgallate, heptyl 3-nitro tribenzoylgallate, heptyl 4-nitro tribenzoylgallate, heptyl 2-chloro tribenzoylgallate, heptyl 3-chloro tribenzoylgallate, heptyl 4-chloro tribenzoylgallate, heptyl 2-bromo tribenzoylgallate, heptyl 3-bromo tribenzoylgallate, heptyl 4-bromo tribenzoylgallate, heptyl 2-fluoro tribenzoylgallate, heptyl 3-fluoro tribenzoylgallate, heptyl 4-fluoro tribenzoylgallate, heptyl 2-methoxy tribenzoylgallate, heptyl 3-methoxy tribenzoylgallate, heptyl 4-methoxy tribenzoylgallate, heptyl 2-hydroxy tribenzoylgallate, heptyl 3-hydroxy tribenzoylgallate, heptyl 4-hydroxy tribenzoylgallate, heptyl 2-methyl tribenzoylgallate, heptyl 3-methyl tribenzoylgallate, heptyl 4-methyl tribenzoylgallate, heptyl 2-acetyl tribenzoylgallate, heptyl 3-acetyl tribenzoylgallate, heptyl 4-acetyl tribenzoylgallate.

In addition, this invention describes the compounds corresponding to the following general formula:

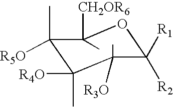

where R3, R4, R5, and/or R6 represent a galloyl group and/or H RI represents a hydrogen or hydroxyl or galloyl group, and/or R2 represent a hydrogen or hydroxyl or galloyl group, and their preparation procedures. More specifically, merely by way of illustration and without limitation, the compounds are the following: 1,2,3,4,6,-penta-O-galloyl-α-D-glucopyranose, 1,2,3,4,6,-penta-O-galloyl-β-D-glucopyranose, 2,3,4,6,-tetra-O-galloyl-D-glucopyranose.

### EXAMPLE 15

Equivalent preparation procedures to obtain all of the above-described acylated derivatives of gallic acid:

Procedure for preparing triacetylgallic acid: A sample of 1 g gallic acid is dissolved in 600 ml analytical grade dichloromethane cooled to 0° C. and 1 g 4-dimethylaminopyridine (DMPA) is added, while stirring, as a catalyst. Then an equimolar quantity plus a slight excess of acetyl chloride is added drop by drop to the resulting solution while stirring and the mixture is stirred for 24 hours at 0° C. The reaction mixture is washed with a 5% HCl solution, dried with anhydrous sodium sulfate and filtered, obtaining triacetylgallic acid by evaporation of the solvent. The purity of the product is checked by thin-layer chromatography using silica gel. The product is spectroscopically characterized (FTIR, UV, NMR, EM).

### EXAMPLE 16

Procedure for preparing the propanoyl, butanoyl, pentanoyl, hexanoyl, heptanoyl, and benzoyl chloride derivatives and substituted benzoyl chlorides in the aromatic ring of gallic acid using the same procedure as described in example 15.

### EXAMPLE 17

Equivalent preparation procedures to obtain all of the above-described ester derivatives of gallic acid:

Procedure for preparing methyl, ethyl, propyl, butyl, pentyl, hexyl, heptyl, benzyl, and substituted benzyl esters of gallic acid: A sample of 1 g gallic acid is dissolved in 50 ml analytical grade methanol and a catalytic quantity of H₂SO₄ is added with stirring. The reaction mixture is heated under reflux and the reaction is continued by thin layer chromatography (TLC). Methyl gallate is obtained by elimination of the solvent. The product is spectroscopically characterized (FTIR, UV, NRM, EM).

The same procedure is followed to obtain ethyl gallate, propyl gallate, and butyl gallate. For pentyl, hexyl, heptyl, benzyl and substituted benzyl gallates, a separation stage by means of silica gel column chromatography is used prior to the spectroscopic characterization.

### EXAMPLE ˜

Equivalent preparation procedures to obtain all of the above-described acylated derivatives of gallic acid esters:

Procedure for preparing methyl triacetylgallate, methyl tripropionylgallate, methyl tributanoylgallate, methyl tripentanoylgallate, methyl trihexanoylgallate, methyl triheptanoylgallate, methyl tribenzoylgallate and methyl (substituted) tribenzoylgallates: A sample of 1 g methyl gallate is dissolved in 600 ml analytical grade dichloromethane cooled to 0° C. and 1 g 4-dimethylaminopyridine (DMPA) is added as a catalyst with stirring. Then an equimolar quantity plus a slight excess of acetyl chloride is added drop by drop to the resulting solution while stirring and the mixture is stirred for 24 hours at 0° C. The reaction mixture is washed with a 5% HCl solution, dried with anhydrous sodium sulfate and filtered, obtaining methyl triacetylgallate by evaporation of the solvent. The purity of the product is checked by thin layer chromatography using silica gel. The product is spectroscopically characterized (FTIR, UV, NMR, EM).

The same procedure is used for ethyl, propyl, butyl, pentyl, hexyl, heptyl, benzyl, and substituted benzyl gallate.

### EXAMPLE 19

Procedure for preparing 2,3,4,6,-tetra-O-galloyl-D-glucopyranose: A suspension of the benzoylated derivative (0.20 mmol), 10% Pd/C (0.20 g), and THF (10 ml) is degasified three times with argon and treated with hydrogen at 40° C. for 24 hours. The reaction mixture is allowed to cool to room temperature; the solid is filtered over Celite and the Celite is washed with 30 ml analytical grade acetone. The combined organic phases are concentrated under vacuum to yield a yellow oil. Reverse-phase chromatography (water:MeOH, 80:20) yields the mixture of α and β anomers in a 90% yield as a yellow powder. The compound is characterized by spectroscopic methods.

### EXAMPLE 20

Prrocedure for preparing 1,2,3,4,6,-penta-O-galloyl-β-D-glucopyranose: A suspension of the benzoylated derivative (0.06 mmol) in 20 ml dry THF is treated with hydrogen following the method described above to yield an oil. Reverse-phase chromatography (water:MeOH, 80:50) yields the β anomers in a 90% yield as a yellow powder. The compound is characterized by spectroscopic methods.

### EXAMPLE 21

Procedure for preparing 1,2,3,4,6,-penta-O-galloyl-α-D-glucopyranose: The synthesis of this compound is produced using the benzoylated derivative under conditions and proportions identical to those used in the previous example.

The extracts obtained are submitted for biological studies, especially in rats, as follows:

Study of [the Effect of] the Extract on Sexual Activity

The effect of the extracts present in the compositions on the sexual activity of male rats was studied in a mating test. This test, which makes it possible to study the effect of various drugs on sexual behavior, consists of measuring a set of variables and analyzing them for clues as to the relationship between the investigated compositions and sexual function (Drago, F., 1999). The results obtained indicate an increase in mountings before ejaculation and an increase in the number of intromissions before ejaculation and in ejaculatory latency, which indicates the time from the first intromission to ejaculation. This demonstrates the existence of statistically significant effects on the mating behavior of the male rat and a high trend pointing to an increase in libido and/or sexual potency.

Study of [the Effect of] the Extract on Behavior

The effect of the extracts present in the compositions on the behavior of male rats in a rat carrier was studied five minutes after the drugs were administered intraperitoneally. This test makes it possible to evaluate various compounds through the neurobehavioral function of laboratory animals. An evaluation of the different variables showed that the composition does not stimulate the sympathetic or parasympathetic systems. Moreover, the composition produced slight lethargy and a decrease in the reflex activity of the animal, even though the animal remained attentive to external stimuli.

Study of [the Effect of] the Extract on the Vas Deferens

The effect of the extracts in the compositions on the vas deferens of rats was also studied. One of the physiological factors affecting blood pressure is the tone of the vascular smooth muscles maintained by the activity of the sympathetic nervous system, specifically alpha-1-adrenergic receptors, where alpha 1 agonists produce vasoconstriction and therefore an increase in blood pressure (Germany et al., 1992). The presence of alpha-1-adrenergic receptors in the cavernous body of the penis has now been demonstrated (Goepel et al., 1999). The vasa deferentia of rats are rich in alpha-i-adrenergic receptors; therefore, this study made it possible to discover the pharmacological action mechanism of composition B. It was concluded that, along with a non-competitive antagonistic mechanism of the α1 adrenergic receptor, there is another mechanism that can involve inhibition at the purinergic receptor level or an effect at the presynaptic level.

This series of tests confirms the principal pharmacological action of the extracts in the compositions on erectile dysfunction, and also outlines an action mechanism.

In addition, another series of tests was conducted to identify other uses and determine whether this compound has the undesirable side effects associated with the prior art compounds for the treatment of sexual dysfunction.

Evaluation of [the Effect of] the Extract on Blood Pressure, and On Cardiac and Respiratory Rate

Evaluation of [the effects] of the extracts present in the compositions on blood pressure and on cardiac and respiratory rate in rats. This evaluation was made on rats with normal blood pressure. It was concluded that it decreases systolic and diastolic blood pressure and average blood pressure, increases the cardiac rate, and increases the respiratory rate.

Evaluation of the Effect of the Extract on Blood Pressure

Evaluation, through a Doppler probe, of the effect of the extracts in the compositions on the systolic blood pressure in rats with L-NAME hypertension. Composition B significantly decreased systolic blood pressure with respect to the L-NAME control.

The aforementioned studies show that the extracts present in the compositions not only do not have the undesirable side effects (chiefly hypertension) associated with the prior art compounds, but actually produce a hypotensive effect that is highly significant both in itself and in connection with the treatment of erectile dysfunction.

Comparative Analysis of the Relative Potencies of the Extract of the Invention and Aqueous Infusion

The relative potencies of each tested fraction were calculated, determining the percentage ratio of inhibition/weight used in the test (inhibitory activity/mg of extract or infusion). FIG. No. 5 shows the relative potencies of the extracts and the tested infusion with respect to antagonism to the adrenergic contractile response in isolated rat aortas; the extracts being Extract A: 0.2 mg/ml butanolic extract; B: 0.2 mg/ml ethanolic extract; C: 0.2 mg/ml ethyl acetate extract; and D: 0.55 mg/ml aqueous infusion.

As can be observed in FIG. No. 5, the fractions clearly indicate that the 0.2 mg/ml butanolic extract, the 0.2 mg/ml ethanolic extract, and the 0.2 mg/ml ethyl acetate extract have greater activity than the aqueous infusion, which was tested at a concentration that was almost 3 times greater (0.55 mg/ml) than that used for the extracts, yielding a potency level approximately 8 to 11 times greater for the extracts than for the aqueous infusion.

With respect to weight, the activities of the derivatives of the extract show greater potency than the infusion, thus supporting the extraction procedure. In other words, the fractions obtained through the use of solvents considerably exceed the pharmacological activity of the extracts with respect to the aqueous infusion.

Additional studies were conducted, on both humans and animals, of the acute and chronic toxicity of the extracts present in the compositions.

Clinical Test on Erectile and Hypotensive Effect

A basic clinical test was conducted on 30 persons to evaluate the erectile effect and the hypotensive effect of the extracts contained in the compositions. A dose of 100 mg and 200 mg of the extracts contained in compositions A, B, C, and D respectively was taken two to three times a day, and the results of the investigations showed an effectiveness of 80% for the erectile effect and 78.5% for the hypotensive effect with respect to the control. The basic clinical trial was repeated with two populations of 30 persons. In the first population, the dose taken was 400 mg and 500 mg of the ground plant, and the results of the investigations showed an effectiveness of 21% for the erectile effect and 18.2% for the hypotensive effect with respect to the control. In the second population, the daily dose taken was an aqueous infusion prepared from 400 mg and 500 mg of the ground plant. The results of the investigations showed an effectiveness of 27% for the erectile effect and 26.2% for the hypotensive effect with respect to the control.

According to the studies, the extracts contained in the compositions of this invention have a clear pharmacological effect on the treatment of both erectile dysfunction and hypertension. None of the extracts presented the undesirable side effects associated with the prior art compunds.

The compositions of the invention can be administered to a patient in therapeutically effective quantities, normally in the range of approximately 20 mg to 250 mg two to three times a day.

The compositions of the invention are formulated in dosage forms that use the active principles known in prior art. They are administered to patients as such or in combination with pharmaceutically suitable excipients in the form of pills, tablets, or capsules. The choice of suitable excipients for the pharmaceutical compositions is a routine task for those versed in the art.

1. Procedure for obtaining extracts from plants of the genus Geum CHARACTERIZED by the fact that, the whole plant having been ground in a blade grinder:
a) the vegetable matter is suspended in analytical grade dichloromethane and extracted by reflux extraction; the suspension is filtered and the procedure is repeated until total extraction has been achieved; a residue corresponding to composition A is obtained by evaporating the collected extracts;
b) then the vegetable matter which was extracted with dichloromethane is extracted with analytical grade ethanol, with stirring, and the procedure is repeated until an extract is obtained (the vegetable matter extracted with ethanol having been discarded); a viscous syrup that corresponds to composition B is obtained by evaporation of the collected ethanolic extracts;
c) then composition B is mixed with chilled water to prevent decomposition and the mixture is stirred; the suspension obtained is extracted with analytical grade ethyl acetate; the ethyl acetate extracts are collected and dried; the suspension is filtered and washed; a residue corresponding to composition C is obtained by evaporation of the extract;
d) and, lastly, the aqueous phase is extracted with analytical grade butanol; the butanolic extracts are processed in the same way as those from ethyl acetate, obtaining composition D;
and to obtain the fractions of interest, further extractions are carried out with other solvents such as water, water-ethanol, ethanol, acetone, acetone-water, methanol, methanol-water, and supercritical fluid; in addition, extracts equivalent to compositions A, B, C, and D are obtained by freeze-drying or evaporation of the solvents.

2. Procedure for obtaining extracts from plants of the genus Geum as claimed in claim 1, CHARACTERIZED by the fact that, preferably the Geum root with equilibrium moisture content having been ground in a blade grinder:
a) the vegetable matter is suspended in analytical grade dichloromethane and extracted by reflux extraction for 12 to 48 hours; the suspension is filtered and the procedure is repeated four more times; composition A is obtained through vacuum evaporation of the collected extracts;
b) then the vegetable matter extracted with dichloromethane is extracted with analytical grade ethanol for 36 to 56 hours, with magnetic stirring, and the procedure is repeated five more times until an extract is obtained (the vegetable matter completely extracted with ethanol having been discarded); a viscid syrup corresponding to composition B is obtained through vacuum evaporation of the collected ethanolic extracts;
c) then Composition B is mixed with chilled water and stirred for 12 to 36 hours. The suspension obtained is extracted with analytical grade ethyl acetate five times; the ethyl acetate extracts are collected, dried with anhydrous sodium sulfate for 6-24 hours; the suspension is filtered and the sulfate is washed with clean ethyl acetate; by evaporating the extract under vacuum and after keeping it in a vacuum desiccator, a residue correponding to composition C is obtained;
d) and, lastly, the aqueous phase is extracted with analytical grade butanol; the butanolic extracts are processed in the same manner as those of ethyl acetate, obtaining composition D.

3. Procedure for obtaining extracts from plants of the genus Geum as claimed in claim 2, CHARACTERIZED by the fact that, in stages:
a) the vegetable matter is extracted by reflux extraction for 20 to 30 hours; the suspension is filtered and the procedure is repeated three more times;
b) the vegetable matter is extracted for 42 to 52 hours, with magnetic stirring, and the procedure is repeated three more times;
c) then composition B is mixed with chilled water and stirred for 20 to 28 hours; the suspension obtained is extracted between three and five times; the ethyl acetate extracts are collected and dried for 10 to 20 hours; the suspension is filtered and the sulfate is washed; then the extract is evaporated and kept in a vacuum desiccator.

4. Procedure for obtaining extracts from plants of the genus Geum as claimed in claim 3, CHARACTERIZED by the fact that, in stages:
a) the vegetable matter is extracted by reflux extraction for 24 hours;
b) the vegetable matter is extracted for 48 hours, with magnetic stirring, and the procedure is repeated two more times;
c) then composition B is mixed with chilled water and stirred for 24 hours; the suspension obtained is extracted five times; the ethyl acetate extracts are collected and dried for 12 hours; the suspension is filtered and the sulfate is washed; then the extract is evaporated and kept in a vacuum desiccator.

5. Pharmaceutical composition A, CHARACTERIZED by the fact that it comprises at least pentacyclic triterpenes selected from alpha amirine, beta amirine, ursolic acid and oleanolic acid and/or monoterpenes selected from camphor, alpha-pinene and beta-pinene.

6. Pharmaceutical composition A as claimed in claim 5, CHARACTERIZED by the fact that it also comprises aromatic compounds selected from benzophenone, 1,2-dihydro-1-phenylnaphthalene, (1,3-dimethyl-3-butenyl)-benzene, ligustilide, cinnamaldehyde, and benzaldehyde.

7. Pharmaceutical composition A as claimed in claims 5 and 6, CHARACTERIZED by the fact that it also comprises hydrocarbons C₂₃H₄₈ and C₂₅H₅₂ selected from 6-tridecene and 2,6-dimethylheptadecane, simple sugars selected from arabinitol, thritol, and glucitol, vegetable sterols or phytosterols selected from P-sitosterol, campesterol, stigmasterol, brassicasterol, and related phytosterols, saturated and unsaturated fatty acids selected from octadecanoic acid, oleic acid, a-linolenic acid, Z,Z-9,12-octadecanoic acid, and hexadecanoic acid, and acyl glycerols such as glyceryl hexadecanoate.

8. Pharmaceutical composition A as claimed in claims 5, 6, and 7, CHARACTERIZED by the fact that it comprises approximately 30%-50% hydrocarbons, approximately 0.2%-1% aromatic compounds, approximately 10%-20% sugars, saturated and unsaturated fatty acids, acyl glycerols, and approximately 1%-3% pentacyclic triterpenes.

9. Pharmaceutical composition A as claimed in claims 5, 6, 7, and 8, CHARACTERIZED by the fact that it comprises approximately 40% hydrocarbons, approximately 0.5% aromatic compounds, approximately 30% sugars, saturated and unsaturated fatty acids, acyl glycerols, and approximately 2% pentacyclic triterpenes

10. Pharmaceutical composition B, CHARACTERIZED by the fact that it comprises at least gallic acid, its derivatives, sugars, and/or associated salts.

11. Pharmaceutical composition B as claimed in claim 10, CHARACTERIZED by the fact that it also comprises ellagic acid esters.

12. Pharmaceutical composition B as claimed in claims 10 and 11, CHARACTERIZED by the fact that it also comprises dehydro-ellagitannins that are precursors of gallic acid, egallic acid, valoneic acid lactone, and glucose.

13. Pharmaceutical composition B as claimed in claims 10, 11, and 12, CHARACTERIZED by the fact that it also comprises pentacyclic triterpenes selected from ursolic acid and/or oleanolic acid.

14. Pharmaceutical composition B as claimed in claims 10, 11, 12, and 13, CHARACTERIZED by the fact that it also comprises phenols such as 1,2,3-trihydroxybenzene, carbohydrates selected from sucrose, glucose, fructose, arabinose, and xylose, flavonoids selected from quercitrin and quercetin, galloyl glucoses selected from 2,4,6-tri-O-galloyl-D-glucose, 1,3,4,6-tetra-O-galloyl-β-D-glucose, 1,2,4,6-tetra-O-galloyl-β-D-glucose, 1,2,3,4,5-penta-O-galloyl-β-D-glucose.

15. Pharmaceutical composition B as claimed in claims 10, 11, 12, 13, and 14, CHARACTERIZED by the fact that it comprises approximately 30%-50% carbohydrates, approximately 1%-3% gallic acid, approximately 1%-3% ellagic acid esters, approximately 0.2%-1% phenols, approximately 2.5%-3.5% flavonoids, approximately 1%-3% pentacyclic triterpenes, approximately 20%-40% galloyl glucoses, and approximately 10%-30% dehydro-ellagitannins.

16. Pharmaceutical composition B as claimed in claims 10, 11, 12, 13, 14, and 15, CHARACTERIZED by the fact that it comprises approximately 40% carbohydrates, approximately 2% gallic acid, approximately 2% ellagic acid esters, approximately 0.5% phenols, approximately 3.5% flavonoids, approximately 2% pentacyclic triterpenes, approximately 30% galloyl glucoses, and approximately 20% dehydro-ellagitannins.

17. Pharmaceutical composition B as claimed in claims 10, 11, 12, 13, 14, 15, and 16, CHARACTERIZED by the fact that it contains mixtures of compositions A, C, and D.

18. Pharmaceutical composition C, CHARACTERIZED by the fact that it comprises at least gallic acid, its derivatives, sugars, and/or associated salts.

19. Pharmaceutical composition C as claimed in claim 18, CHARACTERIZED by the fact that it also comprises ellagic acid esters.

20. Pharmaceutical composition C as claimed in claims 18 and 19, CHARACTERIZED by the fact that it also comprises pentacyclic triterpenes selected from ursolic acid and/or oleanolic acid.

21. Pharmaceutical composition C as claimed in claims 18, 19, and 20, CHARACTERIZED by the fact that it also comprises phenols such as 1,2,3-trihydroxybenzene, flavonoids selected from quercitrin and quercetin, galloyl glucoses selected from 2,4,6-tri-O-galloyl-D-glucose, 1,3,4,6-tetra-O-galloyl-β-D-glucose, 1,2,4,6-tetra-O-galloyl-β-D-glucose, 1,2,3,4,6-penta-O-galloyl-β-D-glucose, and 1,2,3,4,5-penta-O-galloyl-β-D-glucose.

22. Pharmaceutical composition C as claimed in claims 18, 19, 20, and 21, CHARACTERIZED by the fact that it comprises approximately 1%-3% gallic acid, approximately 1%-3% ellagic acid esters, approximately 0.2%-1% phenols, approximately 2.5%-4.5% flavonoids, approximately 1%-3% pentacyclic triterpenes, approximately 20%-40% galloyl glucoses.

23. Pharmaceutical composition C as claimed in claims 18, 19, 20, 21, and 22, CHARACTERIZED by the fact that it comprises approximately 2% gallic acid, approximately 2% ellagic acid esters, approximately 0.5% phenols, approximately 3.5% flavonoids, approximately 2% pentacyclic triterpenes, and approximately 30% galloyl glucoses.

24. Pharmaceutical composition D, CHARACTERIZED by the fact that it comprises at least approximately 10%-30% dehydro-ellagitannins that are precursors of gallic acid, egallic acid, valoneic acid lactone, and glucose.

25. Pharmaceutical compositions A, B, C and D as claimed in claims 5 through 24, CHARACTERIZED by the fact that they also comprise pyrogallol, ethyl gallate, methyl gallate, triacetylgallic acid, propyl gallate, octyl gallate, 3,4,5-trimethoxybenzoic acid, and tannic acid.

26. Compound CHARACTERIZED by the fact that it corresponds to the general formula: 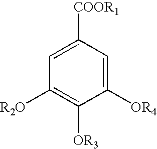
where R1 represents a group selected from acetyl; hydrogen; propionyl; butanoyl; pentanoyl; hexanoyl; heptanoyl; benzoyl; or substituted benzoyl with a nitro, chlorine, bromine, fluorine, methoxyl, methyl, hydroxyl, or acetyl group; methyl; ethyl; propyl; butyl; pentyl; hexyl; heptyl; benzyl; or substituted benzyl with a nitro, chlorine, bromine, fluorine, methoxyl, methyl, hydroxyl or acetyl group; and
R2, R3, and/or R4 represent a group selected from acetyl; hydrogen, propionyl; butanoyl; pentanoyl; hexanoyl; heptanoyl; benzoyl; or substituted benzoyl with a nitro, chlorine, bromine, fluorine, methoxyl, methyl, hydroxyl or acetyl group; methyl; ethyl; propyl; butyl; pentyl; hexyl; heptyl; benzyl; or substituted benzyl with a nitro, chlorine, bromine, fluorine, methyl, methoxyl, hydroxyl, or acetyl group.

27. Compound CHARACTERIZED by the fact that it corresponds to the general formula:
where R3, R4, R5, and/or R6 represent a galloyl group and/or H R1 represents a hydrogen or hydroxyl or galloyl group, and/or R2 represent a hydrogen or hydroxyl or galloyl group.

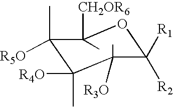

28. Use of pharmaceutical compositions A, B, C, and D as claimed in claims 5 thorough 25 and the compounds of claims 26 and 27, CHARACTERIZED by the fact that they are used to prepare a medication for the treatment of erectile dysfunction in humans and animals.

29. Use of pharmaceutical compositions A, B, C, and D as claimed in claims 5 thorough 25 and the compounds of claims 26 and 27, CHARACTERIZED by the fact that they are used to prepare a medication intended to enhance sexual performance in humans and animals.

30. Use of pharmaceutical compositions A, B, C, and D as claimed in claims 5 thorough 25 and the compounds of claims 26 and 27, CHARACTERIZED by the fact that they are used to prepare a medication intended to treat hypertension in humans and animals.

31. Use of pharmaceutical compositions A, B, C, and D as claimed in claims 5 thorough 25 and the compounds of claims 26 and 27, CHARACTERIZED by the fact that they are used to prepare a medication to be administered orally.

32. Use of pharmaceutical compositions A, B, C, and D as claimed in claims 5 through 25, CHARACTERIZED by the fact that they are used to prepare a medication to be administered in doses of 20to 250 mg two or three times per day. “The following may be redundant and is only a suggestion.” (Carey thinks it is not necessary to include it.):

33. Use of the compounds of claims 26 and 27 CHARACTERIZED by the fact that they are used to prepare a dietary supplement intended to maintain or preserve the normal physiological function of sexual activity.

34. Use of the compounds of claims 26 and 27 CHARACTERIZED by the fact that they are used to prepare a dietary supplement intended to maintain or preserve physiological cardiovascular function.

35. Use of the compounds of claims 26 and 27 CHARACTERIZED by the fact that they are used to prepare a food (dietary) supplement to be administered orally.

36. Use of the compounds of claims 26 and 27 CHARACTERIZED by the fact that they are used to prepare a dietary supplement to be administered in doses of 5 to 200 mg two to three times per day.

37. Use of the compounds of claims 26 and 27 CHARACTERIZED by the fact that they are used to prepare a medication to be administered in doses of 5 to 200 mg two to three times per day.
